(19) United States
(12) Patent Application Publication
(10) Pub. No.: US 2016/0128980 A1
(43) Pub. Date: May 12, 2016

(54) METHODS OF TREATING CKD USING PREDICTORS OF FLUID RETENTION

(71) Applicant: AbbVie Inc., North Chicago, IL (US)

(72) Inventors: Donald K. Kohan, Salt Lake City, UT (US); Hiddo J. Lambers Heerspink, Groningen (NL); Dick de Zeeuw, Groningen (NL); Blai Coll, Highland Park, IL (US); Dennis Andress, Chicago, IL (US)

(21) Appl. No.: 14/934,577

(22) Filed: Nov. 6, 2015

Related U.S. Application Data
(60) Provisional application No. 62/077,108, filed on Nov. 7, 2014.

(51) Int. Cl. A61K 31/4025

(52) U.S. Cl. CPC A61K 31/4025

(57) ABSTRACT

The disclosure relates to methods of treating chronic kidney disease and diabetic nephropathy using predictors of fluid retention to minimize the risk of adverse events. The methods disclosed are particularly useful in treatments involving endothelin receptor antagonists, and more particularly, atrasentan and pharmaceutically acceptable salts thereof.

### PRIORITY

This application claims priority to U.S. Provisional Application No. 62/077,108, filed Nov. 7, 2014, which is hereby incorporated by reference in its entirety.

### FIELD

This disclosure relates generally to methods of treating chronic kidney disease (CKD) based on predictors of fluid retention. Further, this disclosure relates to methods of treating diabetic nephropathy based on predictors of fluid retention.

### BACKGROUND

More than 25% of diabetics have some degree of albuminuria and this leads to the progression of chronic kidney disease. Currently, despite the development of agents that suppress the Renin-Angiotensin Axis System (RAAS) and that slow kidney disease progression, there remains a significant unmet medical need to reduce albuminuria and further slow disease progression. The reduction in albuminuria, as measured by the decrease in the urinary albumin-to-creatinine ratio (UACR), for patients receiving effective doses of RAAS inhibitors like Angiotensin-Converting Enzyme inhibitors (ACEi) or Angiotensin Receptor Blockers (ARBs), is associated with a decrease in the incidence of “hard outcome” renal events like doubling of serum creatinine, time to End-Stage Renal Disease (ESRD) and death.

Although clinical studies have shown that Endothelin Receptor Antagonists (ETRAs) can reduce blood pressure in hypertensive patients with renal disease, ETRAs as well as antagonists of one or both ET_{A} and ET_{B} receptors are known to cause fluid retention. Fluid retention is a common side effect associated with previously studied ETRAs. The observed rate of edema with some previously studied ETRAs resulted in discontinuation of their development for albuminuria or other disease states. Therefore, a balance between desired renoprotection and clinical safety is sought when using ETRAs in a manner that lowers urinary protein excretion while limiting the incidence of peripheral edema and fluid retention.

For example, the ETRA darusentan was studied for the treatment of resistant hypertension. A pivotal double-blind study by Weber (Weber, M., et al., Lancet, 374:1423-31 (2009)), which was conducted in multiple sites worldwide, enrolled 379 individuals with a systolic blood pressure above 140 mmHg who were receiving full doses of at least three blood-pressure-lowering drugs including a diuretic. Patients were randomized to receive either placebo or darusentan (50 mg, 100 or 300 mg) taken once daily. Almost all patients in the study were receiving one of the forms of RAAS inhibitors, nearly three-quarters were receiving a calcium-channel blocker, and approximately two-thirds were receiving a beta-blocker, 99% of patients were receiving full doses of these drugs. In addition, almost all patients were receiving some form of diuretic therapy with the majority (83%) of those on diuretics receiving hydrochlorothiazide at doses of 25 mg per day.

After 14 weeks of treatment, the addition of darusentan was associated with a non-dose-dependent reduction in systolic and diastolic clinic seated blood pressures of about 10 mmHg and 5 mmHg, respectively, compared with placebo treatment. Edema and/or fluid retention was reported in 27% of patients on darusentan and 14% of patients on placebo in the study. Although only four patients (2%) in the combined darusentan treatment groups had to discontinue participation in the study because of fluid retention or peripheral edema, five patients taking darusentan experienced cardiac-related serious adverse events (two patients had myocardial infarction, one patient had atrial fibrillation and two patients had incident congestive heart failure). One sudden death event occurred in the placebo group.

Similar complications were observed in a trial that investigated the use of another ETRA, avosentan, to reduce proteinuria in patients with diabetes. Specifically, the effects of avosentan on the progression of overt diabetic nephropathy were studied by Mann et al. (Mann J., et al., J. Am. Soc., Nephrolo. 21:527-535 (2010)) in a multi-center, multi-national double blind placebo-controlled trial. In this study, 1392 participants with type 2 diabetes were randomly assigned to receive avosentan (25 or 50 mg) or placebo. All patients continued on treatment with ACE inhibitors or ARB agents. After a median follow-up of 4 months, an excess of cardiovascular events with avosentan led to the premature termination of the study. Although avosentan significantly reduced UACR (a median decrease of 44% for avosentan 25 mg, 49% for avosentan 50 mg, compared to only 9.7% for placebo), a significantly increased discontinuation of trial medications due to adverse events occurred for avosentan (19.69% for avosentan 25 mg and 18.2% for avosentan 50 mg, compared to only 1.5% for placebo). Adverse events leading to study dropout for avosentan were predominantly related to fluid overload and congestive heart failure. There were 12 deaths with placebo, 21 deaths with avosentan 25 mg and 17 deaths with avosentan 50 mg.

Bosentan therapy was associated with early worsening of congestive heart failure (CHF) within the first 4-8 weeks in the ENABLE and REACH-1 trials and this was thought to be a consequence of fluid retention. In the EARTH and HEAT-CHF trials, darusentan tended to worsen CHF when given at higher doses.

Intravenous tezosentan at doses greater than 1 mg/hr reduced urine output in patients with acute CHF, thereby limiting clinical efficacy.

Ambrisentan (1-10 mg/day) is associated with edema (25% incidence).

Sitaxsentan at higher doses significantly causes edema; whereas lower doses (100 or 300 mg daily) have less tendency to cause edema.

Atrasentan is a highly potent and selective ETRA that was previously studied for the treatment of prostate cancer. After a detailed evaluation of pre-clinical results, atrasentan was evaluated for the treatment of residual albuminuria. The key objective was to balance systemic effects, which can lead to a significant unacceptable side effects like edema, and efficacy effects on urinary albumin creatinine ratio (UACR). Subsequently, atrasentan was studied in patients with residual albuminuria associated with diabetic nephropathy.

In a double-blind, placebo-controlled, Phase 2 cross-over study in 11 subjects with type 1 diabetes and proteinuria who were not receiving renin-angiotensin system (RAS) inhibitors (referred to herein as Study M96-499), atrasentan 5 mg daily (QD) resulted in a 65% reduction in urinary albumin excretion. Mean arterial blood pressure (BP) was also reduced, however there was only a weak correlation between change in BP and albuminuria reduction. The most commonly experienced treatment-emergent adverse events in the atrasentan group were peripheral edema (64%), pulmonary edema (18%), rhinitis (36%) and headache (18%), whereas in the placebo group there was no peripheral edema, but there were reports of rhinitis (11%) and headache (56%).

As fluid retention and peripheral edema was observed in an oncology program and study M96-499, doses lower than 2.5 mg were contemplated for use to limit edema rates. The doses for a subsequent study were estimated based on receptor binding data while attempting to limit fluid retention and edema.

In a Phase 2a double-blind, randomized, placebo controlled study (referred to herein as M10-815), subjects with type 2 diabetes and albuminuria who were on stable doses of RAS inhibitors were administered atrasentan HCl at 0.25, 0.75 or 1.75 mg QD for 8 weeks. Although, the results of the study demonstrated a reduction in the mean UACR from baseline for subjects taking 0.75 mg QD (42% reduction, 1-sided P=0.023) and 1.75 mg QD (35% reduction, 1-sided P=0.073), a number of subjects experienced mild to moderate peripheral edema—18% in 0.75 mg QD atrasentan and 46% 1.75 mg QD atrasentan dose groups, compared to only 9% in placebo. Only 14% of subjects receiving 0.25 mg QD experienced mild to moderate peripheral edema, but those subjects had a non-significant mean UACR reduction of 21% (P=0.291) compared to subjects receiving placebo (11% reduction). In addition, one subject in the 0.75 mg group who had a predisposition to heart failure suffered an acute heart failure event and was discontinued from the study.

Therefore, a significant benefit would be provided by methods of administering ETRAs in a manner to reduce the risk of edema.

### SUMMARY

Clinical analysis disclosed herein shows that estimated glomerular filtration rate (eGFR), blood pressure, the concentration of glycated hemoglobin (HbA1c), and homeostatic model assessment (HOMA-product) are usable predictors of fluid retention caused by ETRA administration.

The disclosure presents methods of treating chronic kidney disease with an ETRA, such as atrasentan or another selective ET_{A} receptor antagonist, by measuring one or more of eGFR, blood pressure, HbA1c, or HOMA-product in a subject suffering from chronic kidney disease; determining, based on the measurement(s), risk of fluid retention if an ETRA were administered to the subject; and administering the ETRA to the subject if the risk is at an acceptable level.

Additionally, the present disclosure presents methods of treating diabetic nephropathy with an ETRA, such as atrasentan or another ETRA that is a selective ET_{A} receptor antagonist, by measuring one or more of eGFR, blood pressure, HbA1c, or HOMA-product in a subject suffering from diabetic nephropathy; determining, based on the measurement(s), risk of fluid retention if an ETRA were administered to the subject; and administering the ETRA to the subject if the risk is at an acceptable level.

The present disclosure also presents a method of treating chronic kidney disease, diabetic nephropathy or both with endothelin receptor antagonist (ETRA) comprising administering a RAS inhibitor to a subject in need of treatment for chronic kidney disease, diabetic nephropathy or both; measuring one or more of eGFR, blood pressure, HbA1c, or HOMA-product in the subject; determining, based on the measurement, risk of fluid retention if an ETRA were administered to the subject in addition to the RAS inhibitor; and administering the ETRA to the subject if the risk is at an acceptable level. In some embodiments, the RAS inhibitor has been administered to the subject for at least four weeks before the measuring step, and/or the subject has been administered a maximum tolerated labeled daily dose (MTLDD) of a RAS inhibitor for at least four weeks before the measuring step. The foregoing methods can also further comprise the step of adjusting an amount or a frequency of a diuretic already administered to the subject based on the measuring step, and determining the risk of administering the ETRA to the subject based on the measuring step and the adjusting of the diuretic

In the foregoing methods, the risk is risk of fluid retention after two weeks of administering the ETRA to the subject. In some embodiments, the risk of fluid retention is risk of the subject having a weight gain of greater than or equal to 2 kg after administering the ETRA to the subject for two weeks, and/or risk of the subject having a hemoglobin reduction of greater than or equal to 1.3 g/dL after administering the ETRA to the subject for two weeks.

In the foregoing methods, the ETRA can be a selective ET_{A} receptor antagonist, such as atrasentan or a pharmaceutically acceptable salt thereof.

An acceptable risk can be determined by a clinician who weighs the risk of fluid retention against the subject's need for the therapy. The level of risk can be determined manually or automatically such as using an algorithm that calculates a fluid retention risk level using eGFR, blood pressure, HbA1c, and/or HOMA-product as inputs. The level of risk can be determined based on one parameter or on a combination of parameters. For example, the risk level can be determined based on two or more of eGFR, blood pressure, HbA1c, or HOMA-product, or based on eGFR, HbA1c or both.

In some embodiments, the foregoing methods further comprise obtaining a biological sample (for example, urine or blood) from the subject and measuring one of the parameters (for example, eGFR or HbA1c) using the biological sample. In some embodiments, the measurement(s) are compared to predetermined values of eGFR, blood pressure, HbA1c, or HOMA-product which correlate to an acceptable risk of fluid retention. Predetermined values can be acceptable risk level values, unacceptable risk level values, or both.

In some embodiments, when the clinician determines the risk of fluid retention is unacceptable based on the measurement(s) of eGFR, blood pressure, HbA1c, or HOMA-product, the ETRA therapy is not be administered. In some embodiments, if the risk of fluid retention is relatively high, the clinician may adjust the therapy administered to the subject so that an unacceptable risk of fluid retention becomes an acceptable risk, such as by prescribing a diuretic, increasing a dose of a diuretic (such as an amount or frequency), which is already taken by the subject, or changing the diuretic taken by the subject.

### BRIEF DESCRIPTION OF THE DRAWINGS

FIG. 1 depicts changes in UACR, body weight, hemoglobin, and hematocrit from baseline through 12 weeks of atrasentan or placebo administration.

FIG. 2 depicts changes in body weight, hemoglobin, and hematocrit based on whether the subject showed a greater than 30% response in UACR.

### DETAILED DESCRIPTION

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

The terms “administer”, “administering”, “administered” or “administration” refer to any manner of providing a drug (such as, atrasentan or a pharmaceutically acceptable salt thereof) to a subject or patient. Routes of administration can be accomplished through any means known by those skilled in the art. Such means include, but are not limited to, oral, buccal, intravenous, subcutaneous, intramuscular, transdermal, by inhalation and the like.

The term “atrasentan” refers to (2R,3R,4S)-4-(1,3-benzodioxol-5-yl)-1-[2-(dibutylamino)-2-oxoethyl]-2-(4-methoxyphenyl)pyrrolidine-3-carboxylic acid having the structure shown below:

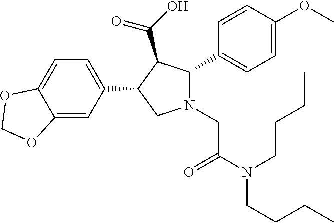 [SMILES: CCCCN(CCCC)C(=O)CN1C[C@H](c2ccc3c(c2)OCO3)[C@@H](C(=O)O)[C@@H]1c1ccc(OC)cc1]

and salts thereof such as the HCl salt of atrasentan. Methods for making atrasentan are described, for example, in U.S. Pat. Nos. 6,380,241, 6,946,481, 7,365,093, 5,731,434, 5,622,971, 6,462,194, 5,767,144, 6,162,927 and 7,208,517, the contents of which are herein incorporated by reference. Treatment regimens for ETRAs with angiotensin converting enzyme (ACE) inhibitors and angiotensin receptor blockers (ARBs) are disclosed in U.S. Pat. Nos. 8,623,819 and 8,865,650, which are herein incorporated by reference.

By an “effective amount” or a “therapeutically effective amount” of an active agent is meant a nontoxic but sufficient amount of the active agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by one of ordinary skill in the art using routine experimentation.

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects.

The term “subject” refers to an animal. In one aspect, the animal is a mammal, including a human or non-human. The terms patient and subject may be used interchangeably herein.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

Two Phase 2b, multicenter, prospective, randomized, double-blinded, placebo-controlled, 12 week studies were conducted to evaluate the efficacy and safety of atrasentan compared to placebo in reducing residual albuminuria. Atrasentan was studied at doses of 0.75 mg QD and 1.25 mg QD. The study methodologies were the identical, but for the location. (NCT01356849 in U.S., Canada, and Taiwan; NCT10424319 in Japan). Additional clinical details can be found in Kohan, D E, et al., “Predictors of Atrasentan-Associated Fluid Retention and Change in Albuminuria in Patients with Diabetic Nephropathy,” Clin J Am Soc Nephrol, September 2015, 10:1568-1574, which is hereby incorporated by reference in its entirety.

Observations in body weight changes and hemoglobin changes may be used as surrogate markers for changes in fluid retention.

Demographic parameters were similar between the placebo and atrasentan 0.75 and 1.25 mg/d arms (Table 1). The following abbreviations are used within the table: BNP=B-type natriuretic peptide; DBP=diastolic blood pressure; eGFR=estimated glomerular filtration rate; HbA1c=glycated hemoglobin; RAS=renin-angiotensin system; SBP=systolic blood pressure; UACR=urinary albumin to creatinine ratio. Table 1 is reproduced in part from de Zeeuw D, et al., The endothelin antagonist atrasentan lowers residual albuminuria in patients with type 2 diabetic nephropathy. J Am Soc Nephrol 25: 1083-1093, 2014, which is hereby incorporated by reference in its entirety. Values within Table 1 are mean±standard deviation unless stated otherwise.

TABLE 1

| | | | Atrasentan | | Atrasentan | |
|---|---|---|---|---|---|---|
| | Placebo | | 0.75 mg/d | | 1.25 mg/d | |
| | (n = 50) | | (n = 78) | | (n = 83) | |
| Age, years | 64.3 | (9.0) | 65.0 | (9.8) | 64.5 | (8.8) |
| Sex, n (%) | | | | | | |
| Male | 40 | (80) | 63 | (81) | 57 | (69) |
| Female | 10 | (20) | 15 | (19) | 26 | (31) |
| SBP, mmHg | 136 | (14) | 138 | (14) | 136 | (15) |
| DBP, mmHg | 72 | (10) | 75 | (10) | 74 | (9) |
| eGFR, mL/min/1.73 m² | 49.3 | (13.3) | 47.9 | (14.6) | 50.6 | (13.6) |
| HbA1c, % | 7.4 | (1.3) | 7.5 | (1.5) | 7.7 | (1.4) |
| Weight, kg | 84.3 | (20.2) | 87.1 | (22.1) | 88.3 | (18.4) |
| Hemoglobin, g/dL | 12.7 | (1.8) | 12.9 | (1.5) | 12.9 | (1.8) |
| Hematocrit, % | 38.1 | (5.4) | 38.8 | (4.3) | 38.6 | (5.3) |
| Serum albumin, g/dL | 4.0 | (0.4) | 4.0 | (0.4) | 4.1 | (0.3) |
| BNP, median (Q1-Q3), pg/mL | 31.7 | (18.3-93.0) | 33.0 | (16.0-70.0) | 33.5 | (14.0-64.0) |
| UACR, median (Q1-Q3), mg/g | 671 | (410-1536) | 878 | (515-1682) | 826 | (481-1389) |
| creatinine | | | | | | |
| RAS inhibitors, n (%) | 50 | (100) | 78 | (100) | 83 | (100) |
| Diuretics, n (%) | | | | | | |
| Loop diuretics | 19 | (38) | 29 | (37) | 27 | (33) |
| Thiazides | 29 | (58) | 42 | (54) | 43 | (52) |

Table 2 depicts changes over measured/calculated parameters over time.

TABLE 2

| | | | | Atrasentan | | Atrasentan | |
|---|---|---|---|---|---|---|---|
| | | Placebo | | 0.75 mg/day | | 1.25 mg/day | |
| | Time | | Mean (SD) | | Mean (SD) | | Mean (SD) |
| Variable | Week | n | or median | n | or median | n | or median |
| UACR, median, mg/g | Baseline | 50 | 671 | 78 | 878 | 83 | 826 |
| | 2 | 48 | 696 | 75 | 573 | 82 | 515 |
| | 6 | 48 | 686 | 74 | 636 | 75 | 461 |
| | 12 | 48 | 797 | 70 | 521 | 69 | 470 |
| | Recovery | 45 | 737 | 68 | 1051 | 71 | 727 |
| Hemoglobin, g/dL | Baseline | 50 | 12.7 (1.8) | 78 | 12.9 (1.5) | 83 | 12.9 (1.8) |
| | 2 | 47 | 12.5 (1.9) | 74 | 12.0 (1.5) | 80 | 11.8 (1.8) |
| | 6 | 48 | 12.6 1.9 | 72 | 11.9 1.7 | 73 | 11.6 1.7 |
| | 12 | 47 | 12.6 (2.1) | 69 | 11.8 (1.6) | 68 | 11.8 (1.8) |
| | Recovery | 26 | 12.3 (1.9) | 39 | 12.4 (1.5) | 47 | 12.5 (1.9) |
| BNP, median, pg/mL | Baseline | 50 | 31.7 | 77 | 33.0 | 82 | 33.5 |
| | 12 | 47 | 35.0 | 66 | 33.1 | 67 | 37.0 |
| Weight, kg | Baseline | 50 | 84.3 (20.2) | 78 | 87.1 (22.1) | 83 | 88.3 (18.4) |
| | 2 | 48 | 83.2 (19.6) | 77 | 88.0 (22.2) | 82 | 89.4 (18.7) |
| | 6 | 48 | 83.3 (19.6) | 74 | 87.3 (21.8) | 75 | 89.6 (19.0) |
| | 12 | 48 | 82.8 (18.6) | 70 | 87.3 (22.3) | 69 | 89.0 (19.2) |
| | Recovery | 44 | 83 (20) | 61 | 85 (23) | 70 | 88 (19) |

Table 2 shows that body weight increased by approximately 1 kg after 2 weeks of treatment compared with a decrease of approximately 1 kg in the placebo group. Although weight declined 1-2 kg during the 30-day recovery period in the atrasentan-treated groups, weight was unchanged in the placebo group. Hemoglobin (Hb) decreased by approximately 1 g/dl in both atrasentan groups after 2 weeks of treatment, and these reductions persisted throughout the treatment period. Hb normalized by 30 days after treatment discontinuation, suggesting that the atrasentan-associated decrease in Hb was caused by hemodilution. Despite the gain in weight in patients who received atrasentan, no significant change was observed in B-natriuretic peptide (BNP). Changes in the diuretic dose were similar were similar among treatment groups throughout the study (4%, 5%, and 8% for the placebo, 0.75 mg/day, and 1.25 mg/day groups, respectively).

Table 3 shows a list of potential independent baseline predictors of changes in weight and hemoglobin after two weeks of atrasentan therapy.

TABLE 3

| | Linear regression | | Logistic regression | |
|---|---|---|---|---|
| | Coefficient | P- | Odds ratio | |
| Variable | (95% CI) | value | (95% CI) | P-value |

Weight response ≧2 kg weight gain

| | | | | | |
|---|---|---|---|---|---|
| Atrasentan (0.75 vs. placebo) | 0.8 | (0.3-1.3) | 0.001 | 3.0 (1.0-8.5) | 0.04 |
| Atrasentan (1.25 vs. placebo) | 1.3 | (0.8-1.7) | <0.001 | 6.6 (2.3-18.6) | <0.001 |
| eGFR (10 mL/min/1.73 m²) | −0.2 | (−0.3-−0.1) | 0.002 | 0.7 (0.4-1.0) | 0.007 |
| HbA1c (%) | 0.2 | (0.1-0.4) | 0.002 | 1.7 (1.3-2.3) | <0.001 |
| Systolic BP (10 mmHg) | 0.1 | (0.0-0.3) | 0.05 | — | |
| HOMA product (log) | −0.2 | (−0.4-−0.1) | 0.007 | 0.7 (0.5-1.0) | 0.03 |

Hemoglobin response ≧1.3 g/dL hemoglobin fall

| | | | | | |
|---|---|---|---|---|---|
| Atrasentan (0.75 vs. placebo) | −0.7 | (−0.9-−0.5) | <0.001 | — | |
| Atrasentan (1.25 vs. placebo) | −1.0 | (−1.2-−0.8) | <0.001 | 5.6 (2.5-12.7) | <0.001 |
| eGFR (10 mL/min/1.73 m²) | 0.1 | (0.1-0.2) | <0.001 | 0.6 (0.2-0.9) | 0.001 |
| Hemoglobin (g/dL) | −0.1 | (−0.1-0.0) | <0.001 | 1.4 (1.1-1.8) | 0.003 |
| Weight (10 kg) | 0.1 | (0.0-0.1) | 0.01 | 0.9 (0.8-1.0) | 0.05 |

The abbreviations used in Table 3 include BP=blood pressure; eGFR=estimated glomerular filtration rate; HbA1c=glycated hemoglobin; HOMA=homeostatic model assessment. The following covariates were included in the initial backward selection model: treatment assignment, age, gender, body weight, Hb, eGFR, albuminuria, systolic blood pressure (BP), eGFR, log transformed homeostatic metabolic assessment (HOMA) product, log-transformed B-type natriuretic peptide (BNP), thiazide and loop-diuretic use. Systolic blood pressure and atrasentan dose were not included in the final logistic regression models for body weight and hemoglobin, respectively.

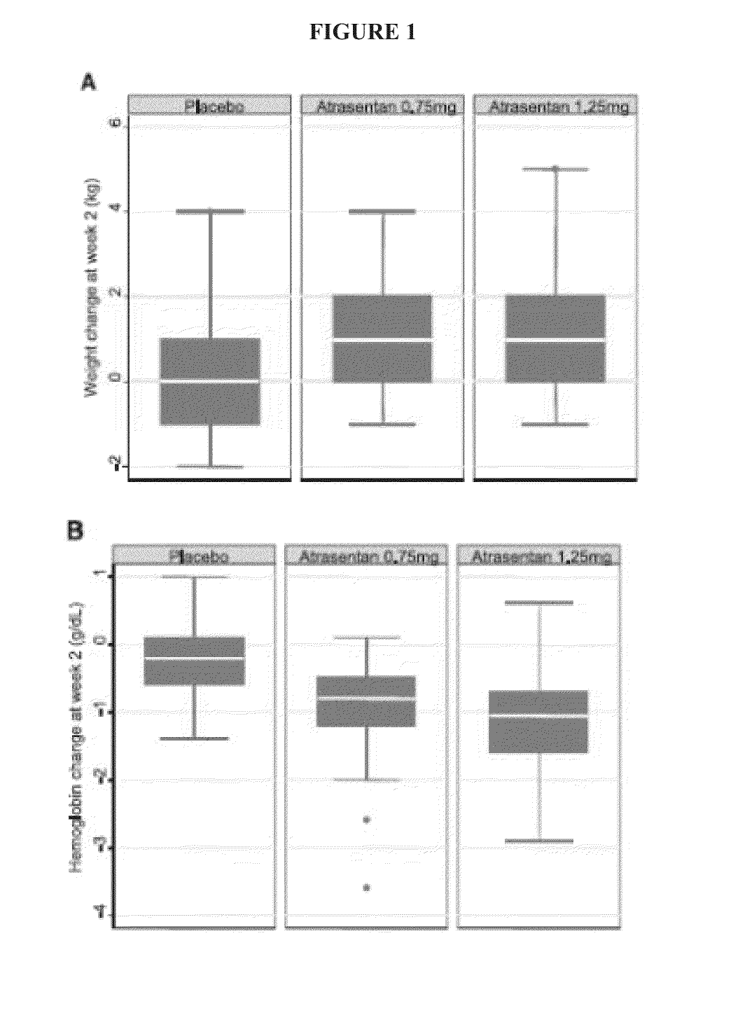

Table 3 confirms that baseline predictors of weight gain after 2 weeks of atrasenttan treatment include an atrasentan dose of 0.75 or 1.25 mg/day versus placebo, lower eGFR, higher glycated hemoglobin (HbA1c), higher systolic BP, and lower HOMA product. For each 10 mL/min lower baseline eGFR, body weight was higher by 0.2 (0.1-0.3) kg. For each percentage lower HbA1c, body weight increased 0.2 (0.1-0.4) kg. For each 10 mmHg lower baseline systolic BP, body weight was higher by 0.1 (0.0-0.3) kg. Logistic regression analysis of factors predicting a greater or equal to 2 kg weight gain (upper quartile of distribution) shows the odds for a greater or equal to 2 kg weight gain were 3.0 (1.0-8.5) and 6.6 (2.3-18.6) times higher for atrasentan 0.75 and 1.25 mg/day groups, respectively. (FIG. 1, Panel A). Determinants in the logistic regression were similar to those in the linear regression model.

Table 3 also discloses the baseline predictors of Hb change after 2 weeks of atrasentan treatment included an atrasentan dose of 0.75 or 1.25 mg/day versus placebo, eGFR, Hb, and weight. Logistic regression analysis of factors predicting a greater or equal to 1.3 g/dl fall in Hb (upper qartile of distribution of combined atrasentan 0.75 and 1.25 mg/day groups) showed an increase in the odds of 5.6 (2.5-12.7) fold with atrasentan 1.25 mg/day versus placebo (but no significant association with 0.75 mg/day group) and 0.6 (0.2-0.9) fold for each 10 ml/min lower baseline eGFR. Small but significant associations with baseline weight and Hb were also observed. Baseline BNP was not associated with changes in body weight or Hb.

Based on this analysis, risk of fluid retention if an ETRA, such as atrasentan, were administered to a subject can be predicted based on measuring one or more of eGFR, blood pressure, HbA1c, or HOMA-product. A clinician can use the measurement to determine if the risk of fluid retention for a particular subject is acceptable before the ETRA is prescribed or administered.

Table 4 shows the correlation between changes in hemoglobin and weight with urinary albumin to creatinine ratio change after 2 weeks of placebo or atrasentan treatment.

TABLE 4

| Variable | Pearson correlation | R² |
|---|---|---|
| Placebo | | |
| Hemoglobin (g/dL) | −0.05 | <0.01 |
| Body weight (kg) | −0.06 | <0.01 |
| Atrasentan 0.75 mg/d | | |
| Hemoglobin (g/dL) | 0.14 | 0.02 |
| Body weight (kg) | −0.26 | 0.07 |
| Atrasentan 1.25 mg/d | | |
| Hemoglobin (g/dL) | 0.18 | 0.037 |
| Body weight (kg) | −0.07 | <0.01 |

The atrasentan dose and eGFR were predictors of week 2 changes in hemoglobin. Moreover, baseline hemoglobin and body weight predicted week 2 changes in hemoglobin.

Weak correlations were observed between week 2 changes in body weight and hemoglobin with changes in albuminuria. R² values ranged between 0.005 and 0.07 suggesting that only 0.5% to 7% of the variability in albuminuria response is explained by changes in body weight. No correlation was detected between changes in UACR and body weight. And no correlation was seen between change in UACR and body weight at week 2 in subjects taking placebo or atrasentan 0.75 or 1.25 mg/day. R² values show that 4% to 6.9% of the variability in the albuminuria response was accounted for by the response in Hb.

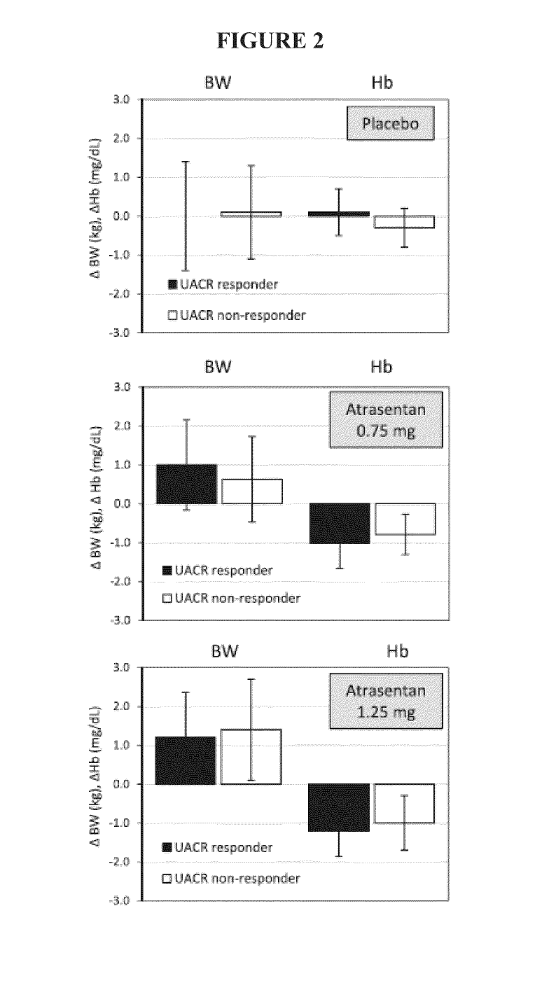

Changes in body weight or Hb were compared between UACR responders (>30% reduction in UACR) and non-responders (<30% reduction in UACR) after treatment with atrasentan for 2 weeks. No difference was detected between UACR responders and non-responders in changes in body weight or hemoglobin, in subjects receiving either atrasentan dose (See FIG. 2).

These data taken together show atrasentan-induced fluid retention is predicted by initial eGFR, blood pressure, and glucose control in patients with type 2 diabetic nephropathy. UACR reduction is not a predictor of fluid retention. Thus, it was surprising and unpredictable as to which baseline measurements are predictors of fluid retention. Moreover, the fluid retention observed with atrasentan treatment is not significantly correlated with the observed albuminuria-lowering effect.

What is claimed is:

1. A method of treating chronic kidney disease with an endothelin receptor antagonist (ETRA) comprising:
measuring one or more of eGFR, blood pressure, HbA1c, or HOMA-product in a subject suffering from chronic kidney disease;
determining, based on the measurement, risk of fluid retention if an ETRA were administered to the subject; and
administering the ETRA to the subject if the risk is at an acceptable level.

2. The method of claim 1, further comprising adjusting therapy administered to the subject to adjust the risk of fluid retention to an acceptable level.

3. The method of claim 1, wherein the ETRA is a selective ET_{A} receptor antagonist.

4. The method of claim 3, wherein the selective ET_{A} receptor antagonist is atrasentan or a pharmaceutically acceptable salt thereof.

5. A method of treating diabetic nephropathy with an endothelin receptor antagonist (ETRA) comprising:
measuring one or more of eGFR, blood pressure, HbA1c, or HOMA-product in a subject suffering from diabetic nephropathy;
determining, based on the measurement, risk of fluid retention if an ETRA were administered to the subject; and
administering the ETRA to the subject if the risk is at an acceptable level.

6. The method of claim 5, further comprising adjusting therapy administered to the subject to adjust the risk of fluid retention to an acceptable level.

7. The method of claim 5, wherein the ETRA is a selective ET_{A} receptor antagonist.

8. The method of claim 7, wherein the selective ET_{A} receptor antagonist is atrasentan or a pharmaceutically acceptable salt thereof.

9. A method of treating chronic kidney disease, diabetic nephropathy or both with endothelin receptor antagonist (ETRA) comprising:
administering a RAS inhibitor to a subject in need of treatment for chronic kidney disease, diabetic nephropathy or both;
measuring one or more of eGFR, blood pressure, HbA1c, or HOMA-product in the subject;
determining, based on the measurement, risk of fluid retention if an ETRA were administered to the subject in addition to the RAS inhibitor; and
administering the ETRA to the subject if the risk is at an acceptable level.

10. The method of claim 9, wherein the RAS inhibitor has been administered to the subject for at least four weeks before the measuring step.

11. The method of claim 9, wherein the subject has been administered a maximum tolerated labeled daily dose (MTLDD) of a RAS inhibitor for at least four weeks before the measuring step.

12. The method of claim 9, further comprising the step of prescribing a diuretic, or increasing a dose of a diuretic which is already taken by the subject, or changing the diuretic taken by the subject, and determining the risk of administering the ETRA to the subject based on the measuring step and the diuretic prescribed, increased or changed.

13. The method of claim 9, wherein the risk level is determined based on two or more of eGFR, blood pressure, HbA1c, or HOMA-product.

14. The method of claim 9, wherein the risk level is determined based on eGFR, HbA1c or both.

15. The method of claim 9, wherein the risk is risk of fluid retention after two weeks of administering the ETRA to the subject.

16. The method of claim 9, wherein the risk of fluid retention is risk of the subject having a weight gain of greater than or equal to 2 kg after administering the ETRA to the subject for two weeks.

17. The method of claim 9, wherein the risk of fluid retention is risk of the subject having a hemoglobin reduction of greater than or equal to 1.3 g/dL after administering the ETRA to the subject for two weeks.

18. The method of claim 9, wherein the ETRA is a selective ET_{A} receptor antagonist.

19. The method of claim 18, wherein the selective ET_{A} receptor antagonist is atrasentan or a pharmaceutically acceptable salt thereof.
